(19) United States
(12) Patent Application Publication
(10) Pub. No.: US 2014/0275950 A1
(43) Pub. Date: Sep. 18, 2014

(54) IMAGING GUIDEWIRE WITH PRESSURE SENSING

(71) Applicant: Volcano Corporation, San Diego, CA (US)

(72) Inventor: Paul Hoseit, El Dorado Hills, CA (US)

(73) Assignee: VOLCANO CORPORATION, San Diego, CA (US)

(21) Appl. No.: 14/205,726

(22) Filed: Mar. 12, 2014

Related U.S. Application Data
(60) Provisional application No. 61/778,796, filed on Mar. 13, 2013.
(60) Provisional application No. 61/787,403, filed on Mar. 15, 2013.

(51) Int. Cl. A61B 5/00

(52) U.S. Cl. CPC A61B 5/0095
USPC 600/407

(57) ABSTRACT

An intravascular element, for example a guidewire, configured for pressure measurements and imaging within a patient. In an embodiment, the invention uses a system of optical fibers and photoabsorptive and/or photoreflective materials to make intravascular ultrasound (IVUS) measurements. In an embodiment, the invention uses microfabricated pressure sensors to measure fluidic pressure adjacent to the element, such as the pressure of the blood within the vasculature. In a clinical setting, the invention can provide a surgeon with critical information about pressure, tissue composition, and luminal area while also reducing the time for procedures.

### RELATED APPLICATIONS

This application claims priority to U.S. Provisional Application No. 61/778,796, filed Mar. 13, 2013 and U.S. Provisional Application No. 61/787,403, filed Mar. 15, 2013, both of which are incorporated herein by reference in their entireties.

### FIELD OF THE INVENTION

The invention relates to intravascular devices that provide multiple diagnostic functions. In particular, the invention relates to guidewires capable of intravascular ultrasound (IVUS) imaging and pressure measurements.

### BACKGROUND

Access guidewires are known medical devices used in the vasculature or other anatomical passageway to act as a guide for other devices, e.g., a catheter. Typically, the guidewire is inserted into an artery or vein and guided through the vasculature under fluoroscopy (real time x-ray imaging) to the location of interest. In some procedures one or more devices are delivered over the guide wire to diagnose, image, or treat the condition.

Crossing guidewires are also known medical devices used in the vasculature or other anatomical passageway, however they are designed to pass through and/or around blockages or narrowed passages in the anatomical passageway, hence the name “crossing.” Crossing guidewires are typically stiffer than access guidewires to provide better tracking and to deliver lateral force. Crossing guidewires are also guided using fluoroscopy. Both access and crossing guidewires can be collectively referred to as “guidewires.”

Advances in materials and miniaturization have made it possible to include sensors on guidewires, such as pressure and flow sensors. For example, the FLOWIRE® Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. These improvements have greatly improved patient care because it is now possible to obtain relevant clinical information during guidewire placement, or during a crossing procedure.

In particular, a pressure-sensing guidewire can be used to evaluate the severity of an occlusion that has been identified with fluoroscopy. If there is a marked pressure increase when crossing an arterial thrombus (or a marked pressure decrease when crossing a venous thrombus), it is likely that the occlusion is severe and causing stress on the cardiovascular system. Such stress can lead to a heart attack or stroke. Additionally, in the event there are multiple occlusions visible with fluoroscopy, pressure measurements help a cardiologist to decide which to treat or image.

In most instances, once an occlusion has been identified as severe, an imaging catheter is delivered along the guidewire in order to evaluate the occlusion. The imaging can be visible, intravascular ultrasound (IVUS), optical coherence tomography (OCT), intravascular magnetic resonance imaging (IVMRI), or other forms of imaging. For example, it is possible to measure the size and composition of the occlusion with IVUS. This information can be used to guide clinical decisions, such as whether the occlusion should be treated with an antithrombus therapeutic or a stent.

While it is possible to obtain the needed information by placing an imaging catheter on the guidewire, it would be preferable if the guidewire, itself, had imaging capabilities, thus reducing allowing a physician to quickly evaluate the need for intervention. Additionally, each catheter exchange increases the length of a procedure while subjecting the patient to additional risks, such as arterial or venous perforation or dislodgement of thrombus.

### SUMMARY

The invention is an intravascular element, e.g., a guidewire, capable of both pressure measurements and imaging. Using a system of optical fibers and photoabsorptive and/or photoreflective materials, the guidewire is capable of making intravascular ultrasound (IVUS) measurements. Additionally, using microfabricated pressure sensors, the guidewire can measure a fluidic pressure, such as the pressure of the blood within the vasculature. The disclosed invention will improve interventional evaluation by providing a physician with critical information about pressure, composition, and luminal area while also reducing the time for procedures.

An additional benefit of the invention is that it allows simultaneous pressure and image evaluation in lumen that are too small for dual purpose catheters, i.e., the instrument more typically used to image vasculature. Ideally, a guidewire of the invention is small, on the order of 1 mm or smaller, allowing the guidewire to be placed throughout the vasculature, as well as the lymphatic, urological, and reproductive systems. Because of this versatility, the guidewire can be used to treat a number of organs, such as the kidneys, lungs, brain, heart, pancreas, ovaries, or testes. Combined pressure and image measurements can be particularly useful in evaluating peripheral arteries, where occlusion are difficult to evaluate.

Additionally, because therapeutic catheters can be used in conjunction with guidewires of the invention, the guidewire can be left in place during the procedure. This allows imaging and characterizing of the interventional area before and after therapy or other procedure, e.g., aspirations. Accordingly, procedure times are shortened, resulting in a reduction of the amount anesthesia, contrast, and x-rays to which a patient is exposed. For example, in an endovascular procedure, the guidewire can be placed once using angiography, the treatment site imaged using the guidewire, a therapy administered, and the treatment site subsequently re-imaged with the guidewire to confirm the results of the treatment.

The invention achieves its versatility by using a system of optical fibers bundled to a core. The design makes efficient use of optical Bragg gratings that work as partially- or fully-reflective wavelength-selective elements. One portion of the fibers are coupled to photoacoustic transducers that convert electromagnetic radiation into acoustic energy, and one portion of the fibers are coupled to acoustic-sensing materials, for example photoreflective material or a strain-gauge type configurations. The invention additionally benefits from the use of miniaturized pressure sensors comprising a diaphragm and a piezoresitive element. In addition to being small enough to fit comfortably within a guidewire, the pressure sensors are also robust and able to interact with a variety of fluids.

In an embodiment, the invention is an intravascular element including a first optical fiber having a first blazed Bragg grating, a photoabsorptive member, and a sensor. The first blazed Bragg grating is designed to be at least partially reflective of a first wavelength. The photoabsorptive member absorbs the first wavelength and is in photocommunication with the first blazed Bragg grating. As discussed above, the sensor includes a diaphragm and a piezoresistive element. The invention additionally lends itself to the disclosed methods of treating a subject, including imaging a subject with acoustic energy produced from a guidewire, and measuring a fluidic pressure with a sensor coupled to the guidewire.

### BRIEF DESCRIPTION OF THE FIGURES

FIG. 1A depicts a distal end of an embodiment of a guidewire having a pressure sensor integrated into the distal tip;

FIG. 1B depicts the simultaneous or sequential delivery and reception of acoustic waves (curved lines) from the distal end of the embodiment of a guidewire of FIG. 1A;

FIG. 2A depicts a distal end of an embodiment of a guidewire having a pressure sensor integrated into the body of the guidewire proximate to the distal end;

FIG. 2B depicts the simultaneous or sequential delivery and reception of acoustic waves (curved lines) from the distal end of the embodiment of a guidewire of FIG. 2A;

FIG. 3A depicts a cross-sectional view of the body of the embodiment of a guidewire of FIG. 1A, showing detail of the photoabsorptive/photoreflective material;

FIG. 3B depicts a cross-sectional view of the distal tip of the embodiment of a guidewire of FIG. 1A, showing detail of the pressure sensor;

FIG. 3C depicts a cross-sectional view of the body of the embodiment of a guidewire of FIG. 2A in proximity to the pressure sensor;

FIG. 4 depicts an embodiment of a system for ultrasound imaging and pressure measurement with a guidewire;

FIG. 5 shows an exemplary use of a guidewire of the invention with an aspiration catheter for treating a thrombus;

FIG. 6 depicts the end view of an aspiration catheter used with an embodiment of a guidewire.

### DETAILED DESCRIPTION

The present invention is an intravascular element, e.g., a guidewire, capable of both pressure measurements and imaging. Using a system of optical fibers and photoabsorptive and/or photoreflective materials, the guidewire is capable of making intravascular ultrasound (IVUS) measurements. Additionally, using microfabricated pressure sensors, the guidewire can measure a fluidic pressure, such as the pressure of the blood within the vasculature. The disclosed invention will improve interventional evaluation by providing a physician with critical information about pressure, composition, and luminal area while also reducing the time for procedures.

In an embodiment, the intravascular elements, e.g., guidewires, methods, and systems of the invention are useful for delivering evaluating intravascular structures, delivering therapy, and/or delivering devices, e.g., catheters, for the purpose of advanced evaluation and/or treatment.

Guidewires typically have diameters of 0.010″ to 0.035″, with 0.014″ being the most common. Guidewires (and other intravascular objects) are also sized in units of French, each French being ⅓ of a mm or 0.013″. Guidewire lengths vary up to 400 cm, depending on the anatomy and work flow. The ends of the guidewire are denoted as distal (far from the user, i.e., inside the body) and proximal (near the user, i.e., outside the body). Often a guidewire has a flexible distal tip portion about 3 cm long and a slightly less flexible portion about 30 to 50 cm long leading up to the tip with the remainder of the guidewire being stiffer to assist in maneuvering the guidewire through tortuous vasculature, etc. The tip of a guidewire typically has a stop or a hook to prevent a guided device, e.g., a catheter from passing beyond the distal tip. In some embodiments, the tip can be deformed by a user to produce a desired shape.

Advanced guidewire designs include sensors that measure flow and pressure, among other things. For example, the FLOWIRE® Doppler Guide Wire, available from Volcano Corp. (San Diego, Calif.), has a tip-mounted ultrasound transducer and can be used in all blood vessels, including both coronary and peripheral vessels, to measure blood flow velocities during diagnostic angiography and/or interventional procedures. Additionally, the PRIMEWIRE® pressure guidewire, available from Volcano Corp. (San Diego, Calif.), provides a microfabricated microelectromechanical (MEMS) pressure sensor for measuring pressure environments near the distal tip of the guidewire. Additional details of guidewires having MEMS sensors can be found in U.S. Patent Publication No. 2009/0088650, incorporated herein by reference in its entirety.

The proximal end of a guidewire varies depending upon the complexity of the device. Simple guidewires, used for placement of devices such as catheters, are untethered, i.e., the proximal end does not need to be connected to other equipment. Sensing guidewires, on the other hand, require a signal connection when the sensor is used. The signal connection is typically detachable to facilitate loading/unloading catheters, however it is also possible to load a rapid exchange catheter on a guidewire prior to guidewire insertion. Placement guidewires without tethers are less expensive, and most useful when a procedure requires multiple catheter exchanges, because each catheter can be quickly removed from the guidewire and the next catheter placed on the guidewire.

While not shown in detail in the figures, a sensing guidewire (like the invention) has a tethered proximal end, typically with a detachable connection. As discussed below, guidewires of the invention use optical fibers to supply light to the distal end of the guidewire and to detect returning light. Accordingly, guidewires of the invention have a tether comprising optical fibers and one or more detachable optical couplings. In some embodiments, all of the optical fibers of the guidewire are coupled into a single optical coupling. The tethers may additionally comprise electrical connections, as needed, to produce acoustic energy or to receive acoustic echoes.

Additionally, while not shown in detail in the figures, a guidewire of the invention has a mid-body connecting the proximal and distal ends. The mid-body is typically a length between 50 and 500 cm, typically greater than or equal to 100 cm, typically less than or equal to 400 cm, typically about 200 to 300 cm. The mid-body typically has a core, which is typically a biocompatible and resilient metal wire. The core may comprise multiple strands of metal fiber or the core may be a unitary piece of metal wire. The core is typically constructed from nitinol or stainless steel. As discussed in greater detail below, the mid-body will also comprise a number of optical fibers to deliver light to the distal end of the guidewire and to return reflected light. The optical fibers may be bound to the core with adhesive or fasteners. The optical fibers may be touching the core or the optical fibers may be displaced axially from the core with spacer, typically a resilient polymer. The core and the optical fibers (and optionally spacer) are coated with a coating to help the guidewire pass through an introducer, to pass through the vasculature, and to help delivered devices (e.g., catheter) easily pass over the guidewire. In addition to being both biocompatible and resilient (will not dislodge or flake), the guidewire coating is typically lubricious to reduce the friction between the guidewire and a catheter.

The sensors incorporated into a guidewire of the invention can be of a variety of structures small enough to be incorporated into a guidewire and suitable for pressure sensing in an anatomical environment, e.g., an artery or vein. The guidewire mounted pressure sensor may be, for example, a MEMS sensor manufactured using deep reactive ion etching (DRIE) to form the solid-state sensor rather than previously used mechanical saws. DRIE is a highly anisotropic etch process for creating deep, steep-sided holes and trenches in solid-state device wafers, with aspect ratios of 20:1 or more. DRIE was originally developed for MEMS structures such as cantilever switches and microgears. However, DRIE is also used for producing other devices such as to excavate trenches for high-density capacitors for DRAM. DRIE is capable of fabricating 90° (truly vertical) walls. Using DRIE leads to a number of new pressure sensor designs for intravascular applications wherein the sensor is mounted at a distal end of a pressure measuring coronary guidewire.

The DRIE method for microelectronic production is capable of etching an arbitrary pattern into a surface of a silicon wafer according to a pattern defined by photolithography. The DRIE process on a silicon substrate produces nearly vertical walls having a depth of 100 μm or more. In fact, the DRIE-based etching can be used to etch completely through a 400 μm thick wafer. Photolithography and DRIE can etch patterns with ^{˜}1 μm precision and create features with dimensions of 1 μm or less. DRIE is widely used in silicon wafer processing. When applied to manufacturing intravascular pressure sensors, the DRIE approach facilitates fabricating pressure sensors that are ideally suited for mounting at a distal end of a coronary guidewire.

The following is a listing of improvements arising from using DRIE in fabricating pressure sensors for coronary guidewires: a non-rectangular sensor substrate facilitates cantilevered support of the delicate pressure sensitive region of the sensor chip; a non-rectangular sensor outline facilitates extremely compact sensor mounting in the tip of a guidewire (by re-orienting the sensor substrate); and a set of precision cutouts in a pressure sensor facilitates employing a simplified arrangement for attaching wires to sensor leads and providing strain relief for the lead wire attachments.

Other potentially useful manufacturing features arise from use of a DRIE approach to form the sensor assembly of a pressure sensor wire. For example, the DRIE manufacturing approach facilitates production of multiple sensor chips simultaneously as a sheet. The individual sensors chips are attached to the sheet via tabs. After fabricating the set of sensor chips within the sheet, the tabs are broken to detach the individual chips from the sheet. A variety of attachment modes are possible, including simple ones that are broken by merely flexing the tab, and more complex tabs that are broken by squeezing an attachment structure. The tabs, in each case, are formed through photolithographic patterning and DRIE in a way to ensure that detaching the sensor chips from their silicon wafer support framework does not damage the sensor chips.

A pressure sensor of an intravascular element, e.g., a guidewire, embodying the DRIE fabrication approach, comprises a pressure sensor chip securely mounted within a transition housing, typically located at a coil-to-coil transition near the distal end of a pressure sensor guidewire (See, e.g., FIGS. 2A and 2B). The housing maintains a relatively constant outer profile in the region containing the pressure sensor chip. In some embodiments a cantilevered portion of the sensor chip has a relatively smaller width than a lead portion to which a set of pressure sensor signal lead wires are coupled. In accordance with illustrative embodiments, the sensor chip can be fabricated using DRIE processing to provide an outline of virtually any desired shape—including chips having curved outline edges.

In an embodiment, the pressure sensor comprises resistive elements, whose resistance (conductivity) changes in response to a change in pressure. To assure that a resistance change in meaningful, the sensor can be equipped with two resistive elements; one element increases resistance with a pressure change and a second element decreases resistance with a pressure change. For example, in an embodiment of the present invention each resistive element has a pressure sensitivity (at 100 mmHg, 25 degrees Celsius) of 15-35 μOhms/Ohm/mmHg. By applying a steady current through the resistive elements, pressure changes result in changes in resistance that in turn result in voltage changes across the resistive sensor elements.

Typically the resistive elements are coupled to a diaphragm that changes shape with pressure. By way of example, a vacuum-filled chamber is formed by etching a well or depression in a silicon wafer, then bonding that first silicon wafer to a second silicon wafer under vacuum. Subsequently, the first silicon wafer is thinned by grinding and etching in a known manner to leave just a thin membrane of silicon, the diaphragm covering the pressure reference chamber. Silicon resistors implanted in the diaphragm prior to the wafer bonding stage now become pressure sensitive by virtue of their inherent sensitivity to strain created by pressure induced flexure of the thin diaphragm. Placement and orientation of the resistors according to well established principles can produce resistive elements having either positive or negative response to applied pressure.

When in operation, a common voltage reference is typically provided, from which voltages across the first and second resistive elements are measured. Connections are established by connecting a first terminal of each of the pair of resistive sensor elements of the sensor to the common reference voltage provided by the signal conditioning device. In some embodiments, a differential amplifier within an associated signal conditioning device senses a voltage difference corresponding to the voltages at the second terminal of each resistive sensor element to establish a voltage difference signal. An analog-to-digital converter (“ADC”) within the signal conditioning device converts the amplified analog voltage difference signal into a digital value. The digital value is received by the processor and filtered (e.g. finite impulse response filtered, or “FIR” filtered) in a known manner to render a filtered digital pressure value based upon prior calibration of the sensor. The filtered digital pressure value is then utilized to drive a digital input to a pair of output digital-to-analog converters (“DACs”). The pair of output DACs renders a differential output signal corresponding to an output signal transmitted on the cable to a pressure controller. In some embodiments, the pressure controller also interfaces with imaging processing software and hardware, allowing the physician to monitor the pressure.

In one embodiment, the silicon resistive elements, for example, have temperature sensitivities ranging from about 2.0 to 3.6 mOhm/Ohm/degree C. Because the temperature sensitivities of the resistive elements are not guaranteed to be identical, at least one of the two signal lines carries an independently adjustable current to facilitate temperature compensation of the pressure sensor as well as other characterization-based adjustments applied by the signal conditioning device to provide accurate pressure sensor readings. The separate sensor drive currents facilitate compensating for differences in changes to resistance in the sensor elements over the range of operating temperatures of the sensor 60. Temperature compensation is achieved by adjusting the excitation current driven on at least one of the two excitation lines to the pressure sensor such that the change in voltage across the sensor elements is substantially the same (i.e., within an acceptable error limit) throughout the entire range of operating temperatures. It is noted that the above-described line composition for the cable connector is exemplary. The sensor to which the signal conditioning device is attachable and the composition of the lines between the sensor and signal conditioning device vary in accordance with design considerations and functional requirements associated with alternative embodiments of the invention.

A distal end of an embodiment of a guidewire 100 is depicted in FIG. 1A. The guidewire 100 comprises optical fibers 110. Optical fibers 110 may be constructed from glass, plastic, or fused silica. Optical fibers 110 include blazed Bragg gratings 115 (discussed below). In the embodiment shown in FIG. 1A, the blazed Bragg grating 115 of the optical fiber 110 is in proximity to an ultrasound transducer 120. The ultrasound transducer 120 may also comprise a photoreflective element that is deflected with the receipt of incident acoustic waves. In other embodiments, the ultrasound transducer and photoreflective elements are separate structures, however it is to be understood that ultrasound transducer 120 refers to a stand-alone ultrasound transducer, a combined ultrasound transducer and photoreflective element, or a stand-alone photoreflective element. The guidewire 100 terminates in a tip 150. The core of the guidewire is not shown in FIG. 1A to assist clarity, however, a core is typically present in the guidewire 100, as discussed above.

The guidewires of the invention employ fiber Bragg gratings to couple light into or out of the optical fibers 110. A fiber Bragg grating is a periodic modulation of the index of refraction in a fiber. When the periodicity, d, of the modulation satisfies the Bragg condition (d=nλ/2) for a wavelength 2, that wavelength will be reflected. That is, the fiber Bragg grating acts as a wavelength-selective mirror. The degree of index change and the length of the grating influences the ratio of light reflected to that transmitted through the grating. A review of fiber Bragg gratings can be found at A. Othonos, Rev. Sci. Inst., 68 (12), 4309 (1997), incorporated by reference herein in its entirety. The optical fibers 110 comprise a normal Bragg grating (back reflective—not shown in FIG. 1A) in addition to blazed Bragg gratings (angle reflective) 115. Blazed Bragg gratings are discussed in greater detail in Othonos, referenced above.

As shown in FIG. 1B, the blazed Bragg gratings couple light, 160, from the optical fibers 110, out of the fibers and into an ultrasound transducer 120. The light 160 originates in a light source, discussed in detail below. As shown in FIG. 1B, the light 160 coupled out of the first optical fiber 110 by the blazed Bragg grating 115 will impinge on the ultrasound transducer 120 producing outbound ultrasonic waves 180. The outbound ultrasonic waves 180 are then absorbed, reflected, and scattered by the tissues surrounding the ultrasonic transducer 120. The inbound ultrasonic waves 190, i.e., reflected, etc. are received by the ultrasonic transducer 120, resulting in a deflection of photoreflective materials (not shown). The change in a pathlength between the photoreflective material and the blazed Bragg grating results in a signal that can be used to image the tissue surrounding the device (discussed in detail below). In some embodiments, a similar structure of blazed Bragg gratings 115 and ultrasonic transducers 120 can be used to make Doppler measurements, e.g., of a flowing fluid, e.g., blood.

The ultrasound transducer 120 comprises an optically-absorptive photoacoustic material, which produces ultrasound waves 180 when it absorbs light 160. The optically absorptive photoacoustic material is positioned, with respect to the blazed Bragg grating 115, to receive the optical energy leaving the blazed grating. The optically absorptive photoacoustic material is selected to absorb light 160, and produce and transmit ultrasound or other acoustic waves for acoustic imaging of a region of interest about the distal tip of the guidewire 100. The acoustic waves generated by the photoacoustic material interact with tissues (e.g., vasculature) in the vicinity of the distal end of the guidewire 100, and are reflected back (echoes). The reflected acoustic waves are collected and analyzed to obtain information about the distance from the tissues to the guidewire, or the type of tissue, or other information, such as blood flow or pressure.

As discussed above, the ultrasound transducer 120 may comprise a photoreflective element to receive reflected acoustic waves. The photoreflective member is flexibly resilient, and is displaced by acoustic waves reflected by the tissues. A transparent (or translucent) flexible material is disposed between the optical fiber 110 and the photoreflective material of the ultrasound transducer 120, thereby allowing a deflection in the photoreflective material to change the path length of the light between the optical fiber 110 and the photoreflective material. In alternative embodiments, a void can be left between the optical fiber 110 and the photoreflective material.

In the absence of incident acoustic energy, the photoreflective material will be in a neutral position, providing a baseline path length between the optical fiber 110 and the photoreflective material. Incident light, transmitted via the optical fiber 110, will be reflected from the photoreflective material, and return to a detector at the proximal end of guidewire (not shown) with a characteristic round trip time. The light transmitted via the optical fiber 110 may be the same light as used to produce acoustic energy (discussed above), the same light used to photoactivate therapeutics (discussed above), or a different light (wavelength, pulse frequency, etc.) may be used. When the photoreflective material is deflected, i.e., with the absorbance of incident acoustic waves, the path length between the third optical fiber 110 and the photoreflective material will change, resulting in a measurable change in the properties of the reflected light, as measured by a detector at the proximal end of guidewire (not shown). The change may be a shift in the time of the return trip, or the shift may be an interferometric measurement. The change in the properties of the reflected light can then be analyzed to determine properties of the tissues from which the acoustic waves were reflected.

In preferred embodiments, the incident light 160 is pulsed at a frequency at which the acoustic waves will be produced. Light sources that produce pulses at ultrasonic frequencies, e.g., 1 MHz and greater, are commercially-available, typically solid state lasers. Nonetheless, photoacoustic materials have natural acoustic resonances, and the photoacoustic material will naturally produce a spectrum of acoustic frequencies when the material absorbs the incident light 160 and the photoacoustic material relaxes by producing acoustic waves. If it is desired to rely on the natural frequencies of the photoacoustic material, the incident light 160 may be continuous.

In an embodiment, the photoacoustic material has a thickness in the direction of propagation that increases the efficiency of emission of acoustic energy. In some embodiments, the thickness of the photoacoustic material is selected to be about one fourth of the acoustic wavelength of the material at the desired acoustic frequency (“quarter wave matching”). Providing photoacoustic material with quarter wave matching improves the generation of acoustic energy by the photoacoustic material, resulting in improved ultrasound images. Using the quarter wave matching and sensor shaping techniques, the productivity of the fiber blazed Bragg sensor and photoacoustic materials approaches the productivity of piezoelectric transducers known in the field of ultrasound imaging.

In one embodiment, before the photoacoustic transducer is fabricated, the guidewire 100 is assembled, such as by binding the optical fibers 110 to the core (not shown) and tip 150, and optionally coating the guidewire 100. The photoacoustic transducer 120 is then integrated into the guidewire 100 by etching or grinding a groove in the assembled guidewire 100 above the intended location of the blazed Bragg grating 115 in the first optical fiber 110. As discussed above, the depth of the groove in the assembled guidewire 100 can play a role in the efficiency of the acoustic wave production (e.g., quarter wave matching).

After the photoacoustic transducer 120 location has been defined, the blazed Bragg grating 115 can be added to the first optical fiber 110. In one example, the grating 115 is created using an optical process in which the portion of the first optical fiber 110 is exposed to a carefully controlled pattern of UV radiation that defines the blazed Bragg grating 115. After the blazed Bragg grating is complete, a photoacoustic material is deposited or otherwise added over the blazed Bragg grating 1115 to complete the transducer 120. An exemplary photoacoustic material is pigmented polydimethylsiloxane (PDMS), such as a mixture of PDMS, carbon black, and toluene. The photoacoustic materials may naturally absorb the light 160, or the photoacoustic material may be supplemented with dyes, e.g., organic dyes, or nanomaterials (e.g., quantum dots) that absorb light 160 strongly. The photoacoustic material can also be “tuned” to selectively absorb specific wavelengths by selecting suitable components.

The guidewires of FIGS. 1A and 1B comprise a pressure sensor 130 that may be fabricated using the DRIE methods, discussed above, and has a resistive diaphragm design, as discussed above. In the embodiments of FIGS. 1A and LB, the diaphragm is covered by a highly elastic material (e.g., a soft silicone elastomer dome), incorporated into the tip 150, and capable of transmitting an applied pressure at the guidewire's tip to the encapsulated pressure sensor's diaphragm. An example of such highly elastic materials is a low durometer silicone elastomer such as MED-4905, MED 4930, or similar from NuSil Technology LLC of Carpinteria, Calif. The applied pressure causes a change in the resistance of the piezoresistive elements on the surface of the diaphragm, and the resulting change in resistance is translated into a change in voltage across the piezoresistive elements. In some embodiments, the sensor 130 includes a flattened/tapered core wire soldered to a housing that supports the sensor chip. See FIG. 3B. The housing may be fixedly attached to the head of the guidewire coil in any of a number of ways including soldering, gluing, or even screwing the housing onto the tip. The wires 140 used to power and communicate with the sensor 130 are attached, for example, by running the wires along a core of the guidewire 100.

An alternate embodiment of a guidewire having IVUS and pressure sensing capabilities is shown in FIGS. 2A and 2B. Like FIGS. 1A and 1B, the guidewire 100 comprises a sensor 230, however in FIGS. 2A and 2B, the sensor 230 is exposed to an applied pressure from within the housing of the guidewire. In this embodiment, fluid is allowed to pass through one or more openings 220 of the guidewire coil. A pressure is therefore applied to an exposed surface of the sensor 230, e.g., the diaphragm. In this embodiment, there is no need for cutouts since the contacts are on the surface of the chip that faces the wires of sensor. However, DRIE patterning may be used to create a receptacle (either partial or fully through the sensor) for the wire to ensure proper placement and to provide mechanical support and strain relief for the electrical connections. In the embodiment shown in FIGS. 2A and 2B, the sensor 230 may be incorporated into a cantilevered structure 240 that allows the sensor to “float” inside the guidewire, thereby reducing the likelihood of errant readings due to contact with portions of the guidewire interior.

In another embodiment, not shown in the figures, the optical fibers 110 may be modified to include first and second normal Bragg gratings. These first and second normal Bragg gratings are partially and mostly reflective, respectively, and create a resonant cavity in the optical fiber 110. In the absence of incident acoustic energy, light in the resonant cavity has a characteristic return signature, e.g., an optical decay signal. With the incidence of reflected acoustic energy, the path length and/or path direction of the resonant cavity will be modified, leading to a change in the return signature. By monitoring changes in the return signature, it is possible to determine the timing of reflected acoustic signals, and hence, properties of the tissues from which the acoustic waves were reflected. The detection is similar to known methods of detecting strain or temperature changes with optical fibers.

In one example of operation of this alternate embodiment, light reflected from the blazed Bragg grating 115 excites the photoacoustic material 120 in such a way that the optical energy is efficiently converted to substantially the same acoustic frequency for which the resonant cavity sensor is designed. The blazed Bragg grating 115 and the photoacoustic material 120, in conjunction with the resonant sensor, provide both an acoustic transducer and a receiver, which are harmonized to create an efficient unified optical-to-acoustic-to-optical transmit/receive device. In some embodiments, more than one type of light (e.g., wavelength) can be coupled into the same fiber, allowing one to be used to produce the acoustic wave and another to monitor reflected acoustic waves. In a further example, the optical transmit/receive frequencies are sufficiently different that the reception is not adversely affected by the transmission, and vice-versa.

Cross sectional view of guidewires are shown in FIGS. 3A to 3C. As in FIGS. 1A-2B, the core has been left out for clarity. FIG. 3A shows across section taken at detail AA in FIG. 1A, including optical fibers, 110, ultrasound transducers 120, and signal wire 140. As shown in FIG. 3A, the ultrasound transducer 120 is substantially in communication with the exterior of the guidewire and the respective optical fibers. FIG. 3B shows a cross section taken at detail BB in FIG. 1A, showing the detail of the sensor 130 embedded in the tip 150. As shown in FIG. 3B, the sensor 130 is coupled to cutouts 340 for the signal wire 140 using contacts 320. Finally, as shown in FIG. 3C, across section of the alternative embodiment shown in FIG. 2A shows a sensor 130 interior to the guidewire, floating within a space accessible to the exterior of the guidewire via opening 220. In this design a fluid, e.g., gas, liquid, e.g., blood can communicate with the sensor without needing to transmit the force through another material, e.g., as in FIGS. 1A and 1B.

In preferred embodiments, guidewires of the invention will comprise a plurality of optical fibers as well as arrays of acoustic transducers, acoustic receivers, and lenses for delivering electromagnetic radiation.

The guidewires described will typically be used as part of a system. An exemplary system 600 is shown in FIG. 4. The system includes a guidewire 610 having an optical fibers 614 coupled to the proximal end, allowing a source of light 620 to be coupled into the optical fiber. Of course, multiple optical fibers may be coupled into a single fiber, such as 614, to facilitate signal production and detection. The source light may be coupled or split with fiber couplers, dichroics, and filters as necessary to achieve the desired performance. Furthermore, a particular fiber need not be limited to a single light source, as some fibers can support multiple wavelengths simultaneously and the wavelengths can be separated for analysis using known multiplexing techniques.

The source of light 620 for the system 600 may be any known light source capable of producing light with the desired temporal and frequency characteristics. Source 620 may be, for example, a solid-state laser, a gas laser, a dye laser, or a semiconductor laser. Sources 620 may also be an LED or other broadband source, provided that the source is sufficiently powerful to drive the photoacoustic transducers. In some instances the sources 620 is gated to provide the needed temporal resolution. In other instances, the source 620 inherently provides short pulses of light at the desired frequency, e.g., 20 MHz.

A detector 640, coupled to fiber 616 is used to measure changes to the coupled light to determine how the acoustic environment of the guidewire 610 is changing. The detector may be a photodiode, photomultiplier tube, charge coupled array, microchannel detector, or other suitable detector. The detector may directly observe shifts in return light pulses, e.g., due to deflection of the photoreflective material, or the detector may observe interferometric changes in the returned light due to changes in pathlength or shape. Fourier transformation from time to frequency can also be used to improve the resolution of the detection.

As shown in FIG. 4, a controller 650 will be used to synchronize the source 620 and the detector 640. The controller may maintain system synchronization internally, or the system may be synchronized externally, e.g., by a user. A pressure controller 630 will be used to synchronize measurements with pressure sensor 130 and may also output measured values to the image processors 660 so that pressure values can be displayed in real-time, e.g., color coding on images.

The output of the detector 640 will typically be directed to image processing 660 prior to being output to a display 670 for viewing. The image processing may deconvolve the reflected light to produce distance and/or tissue measurements, and those distance and tissue measurements can be used to produce an image, for example an intravascular ultrasound (IVUS) image. The image processing may additionally include spectral analysis, i.e., examining the energy of the returned acoustic signal at various frequencies. Spectral analysis is useful for determining the nature of the tissue and the presence of foreign objects. A plaque deposit, for example, will typically have a different spectral signature than nearby vascular tissue without such plaque, allowing discrimination between healthy and diseased tissue. Also a metal surface, such as a stent, will have a different spectral signal. Such signal processing may additionally include statistical processing (e.g., averaging, filtering, or the like) of the returned ultrasound signal in the time domain. Other signal processing techniques known in the art of tissue characterization may also be applied.

Other image processing may facilitate use of the images or identification of features of interest. For example, the border of a lumen may be highlighted or plaque deposits may be displayed in a visually different manner (e.g., by assigning plaque deposits a discernible color) than other portions of the image. Other image enhancement techniques known in the art of imaging may also be applied. In a further example, similar techniques can be used to discriminate between vulnerable plaque and other plaque, or to enhance the displayed image by providing visual indicators to assist the user in discriminating between vulnerable and other plaque. Other measurements, such as flow rates or pressure may be displayed using color mapping or by displaying numerical values.

The use of a guidewire 700 of the invention in combination with an aspiration catheter 800 is shown in FIGS. 5 and 6. FIG. 5 illustrates a longitudinal cross-sectional view of a vessel, having vessel walls 720, defining a lumen. In the example described in FIG. 5, the vessel is occluded with a thrombus 740. After entry into the patient, the guidewire 700 is directed past the thrombus, allowing the thrombus, and tissues past the thrombus, to be imaged. Because guidewire 700 includes a pressure sensor, the user can be immediately alerted when the guidewire transitions though or past the thrombus 740. Using guidewire 700 the thrombus 740 can be identified for treatment with a photoactivated therapeutic, e.g., a thrombolytic agent. The aspiration catheter 800, having a lumen 820 (shown in FIG. 6) for following guidewire 700, can be delivered as near as safe to thrombus 740. Once in position, the thrombus 740 can be aspirated via an opening 810 in the aspiration catheter 800. During and after aspiration, the tissue can be monitored using the imaging capabilities of the guidewire 700, discussed above. For example, a reduction in pressure may be indicative that the thrombus 740 has been removed.

While FIG. 5 shows delivery of a therapeutic to a thrombus, it should be realized that the guidewires, methods and systems described are well suited for delivering therapeutics to many types of tissues. For example, an antiangiogenic drug, such as paclitaxel, can be deactivated for transport to the vicinity of a tumor in the lung using a drug delivery catheter with light activation. Once delivered to the tumor, e.g., with a drug delivery catheter, the deactivated therapeutic can be photoactivated, releasing concentrated paclitaxel in a potent form in proximity to the tumor. Thus, only the tumor and the immediately surrounding tissues will be exposed to the powerful antiangiogenic agent.

The guidewires, methods, and systems of the invention may be used in the treatment of a number of disorders in a subject. For example, the guidewires, methods, and systems can be used to treat a variety of vascular diseases, including, but not limited to, atherosclerosis, ischemia, coronary blockages, thrombi, occlusions, stenosis, and aneurysms. The guidewires, methods, and systems can be used to access and treat a large number of locations that are accessible via the vasculature or urological or reproductive tracts. Such locations include the heart, brain, lungs, liver, kidneys, prostate, ovaries, testes, gallbladder, pancreas, and lymph nodes, among other locations. The guidewires, methods, and systems can be used to treat a variety of diseases, including cardiovascular disease, cancer, inflammatory disease (e.g., autoimmune disease, arthritis), pain, and genetic disorders.

Some embodiments described herein can be constructed using a combination of DRIE processing and lapping (to remove excess silicon from the mechanical substrate) or any other wafer thinning method to facilitate fabrication of a frame containing multiple sensor chips attached by thin, so that at no point is there a need to handle a thin, delicate wafer.

Silicon pressure sensors for this coronary guidewire application normally require a built-in reference chamber, since it is impractical to provide an atmospheric pressure reference inside the coronary artery. The reference chamber is typically formed by creating a sandwich of two silicon wafers or of a silicon wafer and a glass wafer. By way of example, a vacuum-filled chamber is formed by etching a well or depression in a first silicon wafer, then bonding that first silicon wafer to a second silicon wafer under vacuum using the silicon fusion bonding method. Subsequently, the first silicon wafer is thinned by grinding and etching in a known manner to leave just a thin membrane of silicon, the diaphragm, covering the pressure reference chamber. Silicon resistors implanted in the diaphragm prior to the wafer bonding stage now become pressure sensitive by virtue of their inherent sensitivity to strain created by pressure induced flexure of the thin diaphragm. Placement and orientation of the resistors according to well established principles can produce resistive elements having either positive or negative response to applied pressure. Once this wafer sandwich is formed with its myriad diaphragms, reference chambers, and piezoresistors, the pressure sensor fabrication is completed by adding metallized bonding pads and patterning the sensor outlines with DRIE.

In accordance with an exemplary method DRIE processing etches the sensor outlines for a set of sensor chip devices on a single silicon wafer (sandwich). In an exemplary embodiment, DRIE is carried out to a depth of approximately 100 μm. During DRIE processing, the wafer is still 400 μm thick, and relatively resistant to breakage. Next, the DRIE processed wafer is mounted in a lapping machine. By way of example, wax secures the wafer to a holder. The wafer is thereafter lapped in a known manner to remove excess wafer material. Once the device has been thinned to the DRIE depth (e.g., 100 μm) the set of solid-state sensor chip become separated from the bulk of the wafer (except for narrow breakable tabs), and are supported primarily by the wax matrix and the holder. Lapping continues until the desired device thickness is achieved (e.g., 75 μm). The individual pressure chip devices are thereafter freed from the holder by soaking in hot water or solvent to melt or dissolve the wax, leaving thin, individual pressure sensor devices behind, attached to a framework by narrow breakable tabs.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. An intravascular element comprising:
a first optical fiber comprising a first blazed Bragg grating, the grating being at least partially reflective of a first wavelength;
a photoabsorptive member that absorbs the first wavelength and is in photocommunication with the first blazed Bragg grating; and
a sensor comprising a diaphragm and a piezoresistive element.

2. The intravascular element of claim 1, wherein the sensor is a fluidic pressure sensor.

3. The intravascular element of claim 2, wherein the sensor is in fluidic communication with the exterior of the intravascular element.

4. The intravascular element of claim 1, wherein the photoabsorptive member is in acoustic communication with the exterior of the intravascular element.

5. The intravascular element of claim 4, wherein photoabsorption of the first wavelength by the photoabsorptive member creates acoustic waves in proximity to the intravascular element.

6. The intravascular element of claim 1, further comprising:
a second optical fiber comprising a second blazed Bragg grating being at least partially reflective of a second wavelength; and
a photoreflective member that reflects the second wavelength and is in photocommunication with the second blazed Bragg grating.

7. The intravascular element of claim 6, wherein acoustic waves in proximity to the intravascular element cause a deflection of the photoreflective member.

8. The intravascular element of claim 7, wherein deflection of the photoreflective member creates a change in a pathlength for the second wavelength between the second blazed Bragg grating and the photoreflective member.

9. The intravascular element of claim 8, wherein the first and second wavelengths are the same.

10. The intravascular element of claim 1, wherein the diameter of the intravascular element is 3 mm (9 French) or less.

11. The intravascular element of claim 1, wherein the intravascular element is a guidewire.

12. A guidewire comprising:
a first optical fiber comprising a first, a second, and a third blazed Bragg grating, the gratings being at least partially reflective of a first wavelength;
a photoabsorptive member that absorbs the first wavelength and is in photocommunication with the first blazed Bragg grating; and
a sensor comprising a diaphragm and a piezoresistive element.

13. The guidewire of claim 12 wherein the first blazed Bragg grating is between the first and third Bragg gratings.

14. The guidewire of claim 12, wherein an optical pathway between the second and third Bragg gratings changes when the guidewire absorbs acoustic energy.

15. A method of evaluating a subject, comprising:
imaging a subject with acoustic energy produced from a guidewire; and
measuring a fluidic pressure with a sensor coupled to the guidewire.

16. The method of claim 15, wherein the guidewire comprises
a first optical fiber comprising a first blazed Bragg grating, the grating being at least partially reflective of a first wavelength;
a photoabsorptive member that absorbs the first wavelength and is in photocommunication with the first blazed Bragg grating; and
a sensor comprising a diaphragm and a piezoresistive element.

17. The method of claim 16, wherein the sensor is a fluidic pressure sensor.

18. The method of claim 16, wherein the guidewire further comprises:
a second optical fiber comprising a second blazed Bragg grating being at least partially reflective of a second wavelength; and
a photoreflective member that reflects the second wavelength and is in photocommunication with the second blazed Bragg grating.

19. The method of claim 15, wherein imaging comprises imaging at least a portion of an anatomical system selected from a cardiovascular system, a lymphatic system, a urological system, or a reproductive system.

20. The method of claim 19, wherein imaging comprises imaging an artery or vein of the cardiovascular system.
